(19) United States
(12) Patent Application Publication
(10) Pub. No.: US 2010/0047310 A1
(43) Pub. Date: Feb. 25, 2010

(54) Bio-acceptable conduits and method providing the same

(75) Inventors: Chien-Chung CHEN, Taipei (TW); Jeng-Chang YANG, Taipei (TW); Suhan LI, Sijhih (TW); En-Sheng KE, Taipei (TW); Yung-Sheng LIN, Sioushuei (TW)

Correspondence Address: BACON & THOMAS, PLLC, 625 SLATERS LANE, FOURTH FLOOR, ALEXANDRIA, VA, 22314-1176, US

(73) Assignee: Taipei Medical University, Taipei (TW)

(21) Appl. No.: 12/458,526

(22) Filed: Jul. 15, 2009

(30) Foreign Application Priority Data
Aug. 21, 2008 (TW) 097131897

(51) Int. Cl. A61F 2/04; C12N 5/06; A61F 2/02; B29C 47/00

(52) U.S. Cl. 424/423; 435/325; 264/465; 606/152; 623/23.71

(57) ABSTRACT

Disclosed is a bio-electrospinning technique for preparing a cell-containing, oriented, continuous tubular scaffold, made of biodegradable polymer, designed for use as a nerve guide conduit (NGC) in nerve regeneration. With a coaxial spinneret, the PC-12 cell medium solution was co-electrospun into a core of tubular fibers, with PLA on the outer shell. The resulted fibers' morphology was characterized via SEM and optical microscopy, and following structural characteristics were found: 1. the larger, hollow fibers had diameters in tenth of microns and wall thicknesses around few microns, 2. an orientation in a preferred direction with the aid of a high-rotating collection device. The fluorescent PC12 cells embedded within the scaffold were cultured and nerve growth factor was added. We observed cells could not only survive the process, but also sustain their viability by undergoing differentiation process, extending neurite along the micro tubular scaffold in the desired direction. All these results demonstrate its potential application for advanced NGC.

### BACKGROUND OF THE INVENTION

1. Field of the Invention

The present invention relates to a nerve guide conduit and the method providing thereof, more particularly, to a bio-acceptable (i.e. biocompatible/biodegradable) nerve guide conduit and the method providing thereof.

2. Description of Related Art

Studies of nerve regeneration, particularly the repair of either damaged peripheral or spinal cord nerves, have drawn tremendous attention in the past few years. This is especially true, as part of the overall concept of regenerative medicine, specifically, tissue engineering. For severe cases of nerve damage, current regeneration strategy calls for nerve grafts, either autografts or artificial nerve guides, which can bridge gaps larger than several centimeters. Considered the gold standard, the auto-graft suffered from limited resources and loss of mobility from donor sites. The synthetic nerve guide conduit, on the other hand, has had several advantages, such as no immunization concern, adequate mechanical properties, unlimited supply, and is hence considered a promising candidate for such surgical needs. Currently, only limited FDA certified commercial nerve guide conduits are available, despite the increasing need for such medical devices. They are all simple, single-lumen constructs with diameters ranging from 2 to 10 millimeters. Material-wise, they are all made of degradable polymers, such as collagen, polylactic acid, and PCL. Improvements of this simple conduit were proposed and studies carried by several research groups. Improvement approaches can be best summarized by the several models proposed. Structure-wise, both Lietz and Bellamkonda proposed models based on the structural elements. On the other hand, Hudson proposed models that emphasized the essential functions of the advanced nerve guide. Based on these models, recent advanced NGC (nerve guide conduit) studies have focused on several distinctions, namely (1) providing more guiding structure, (2) adding chemical cues (such as, NGF, ECM), and (3) integrating all the above factors. Several studies have examined the multi-lumen structure via several processing techniques. Parallel agarose tubes were prepared by freezing the agarose aqueous solution from one side and forcing the alignment of the continuous tubes upward. Without using any toxic chemicals as solvent, the in vitro test demonstrated the fast growth of the neurite in the guiding direction. However, the majority of such approaches called for traditional processes, such as injection molding or simple molding as presented by Moore et al. On the other hand, Hodlock et al injected PLGA/glacial acetic acid into a mold with several arranged stainless steel wires. After removing the acid solvent by freeze dry and then the stainless steel wires, a multi-lumen conduit was obtained. Bender et al prepared PCL nerve guide by coating PCL on PVA core fibers, and then washing out the PVA to make several lumen channels. Huang et al fabricate coaxial stacked nerve conduits through soft lithography and molding processes.

With these efforts, the dimensions of these tube units were in the range of several hundred microns to a few millimeters. There has been little research demonstrating structural units down to a few microns or even in the nanometer range. Li et al prepared a nano-scale patterned plate as the mold for PLLA, rolling up as tubes for NGC and providing 4 to 8 times of the surface area for the cell to attach and grow. Similarly, Papenburg et al reported a phase separation micromolding process to fabricate porous micropatterned 2-D scaffolds.

In terms of tissue engineering, the integration of the scaffold, cell and signal, is crucial for an effective regenerative surgery. Most of the above mentioned scaffolds can only introduce cell and/or signal after the completion of the scaffold, due to the extreme situation during the fabrication process, such as high or low temperature. The seeding of the cell in inner part of scaffold might be difficult, especially for larger objects with finer structural features. It would be very beneficial if cells could be introduced into the scaffold in situ. Finally, the addition of the chemical cues, such as growth factor, could be achieved in a controlled manner by fine tuning the degradation mechanism of biodegradable polymers such as polylactic acid.

### SUMMARY OF THE INVENTION

With bio-electrospinning, we present a novel scaffold approach, especially for an advanced NGC (nerve guide conduit), which not only reduces the dimension of the guiding structural unit for maximum cell attachment and growth, but also effectively integrates all three major components of tissue engineering into one simple, low cost process.

An object of the present invention is to provide a method for producing multifunctional nerve guide conduits, whereby the method of the present invention is able to solve the problems existing in the art such as great complexity of manufacturing procedures, excessive time-consumption, and high-cost.

The method of fabricating nerve guide conduits of the present invention comprises: (A) providing (preparing) an electrospinning device, in which the electrospinning device comprises: a core/shell spinneret having an inner outlet and an outer outlet with that the inner outlet and the outer outlet are coaxial; a first syringe pump connecting to the inner outlet of the core/shell spinneret; a second syringe pump connecting to the outer outlet of the core/shell spinneret; and a collecting unit; (B) feeding the second supplying syringe with a first material, and feeding the first supplying syringe with a supporting solution; (C) electrospinning the first material and the supporting solution by using the electrospinning device to extrude a plurality of hollow conduits, with the supporting solution inside, out from the core/shell spinneret; (D) collecting the hollow conduits by the collecting unit, and arranging the hollow conduits parallelly; and (E) curling up the hollow conduits to provide a nerve guide conduit, wherein the first material is a biodegradable material or a biocompatible material.

Compared with the conventional methods, the method of fabricating nerve guide conduits of the present invention is quite simple. The method of the present invention utilizes an electrospinning method to produce biodegradable/biocompatible hollow conduits, following with curling up the hollow conduits after being parallelly arranged, thus the desired nerve guide conduit is obtained. The present invention is the first one applying an electrospinning method into the fabrication of the multi-tubular nerve guide conduits, and the method of the present invention can largely reduce process time, and improve fabricating efficiency.

The nerve guide conduit of the present invention can be degraded or may not be rejected in vivo because it is made of biodegradable materials or biocompatible materials. Each of the nerve guide conduits of the present invention comprises several hollow conduits that are tube-shaped with through channels in the long axis direction, thus the nerve guide conduit of the present invention has a large surface area for the growth of the cells. Besides, with the characteristic of the material of polylactic acid (PLA) itself and the well molecules arrangement by the electrospinning procedure, the nerve guide conduit of the present invention may have specific piezoelectricity. Therefore, an inner stimulation such as diameter changing of the hollow conduits or an outer stimulation such as ultrasonic waves may enhance electric current for axon growth guidance/induction.

Preferably, the method of the present invention may further comprise a step (C1), after step (C), of washing the hollow conduits with a solvent in order to wash out the supporting solution filled in the hollow conduits. The solvent used is not limited, but preferably is water.

According to the method of the present invention, the first material preferably is, but is not limited to, polylactic acid (PLA), polyglycolic acid (PGA), poly(lactic-co-glycolic acid) (PLGA), polycaprolactone (PCL), collagen, chitosan, polyalkyl acid, alginate, polyamide, or the combinations thereof.

According to the method of the present invention, the supporting solution preferably is, but is not limited to, a solution of poly vinyl pyrrolidone (PVP), poly ethylene oxide (PEO), poly ethylene glycol (PEG), or the combinations thereof.

According to the method of the present invention, the supporting solution may preferably contain at least one cell, the cell preferably being a nerve regeneration cell, but is not limited thereto. The nerve regeneration cell is preferably a neural stem cell, Schwann cell, Satellite Cells, oligodendrocyte, astrocyte, microglia, ependymal cells, or the combinations thereof, but is not limited thereto. Thereby, cells can be delivered into the nerve guide conduit during (not after) the electrospinning process with the supporting solution as a carrier. Accordingly, no excess steps are required for putting cells into the nerve guide conduit after the nerve guide conduit has been formed, the hole processing steps for fabricating a nerve guide conduit containing cells inside are simplified, which cannot be obtained by the conventional methods.

Besides, according to the method of the present invention, at least a growth factor may be preferably added into the first material in the step (B) in order to induce the differentiation of the cell. For the tissue engineering point of view, growth factor/chemical cues is the one of the three major components of the tissue engineering concept. In the following example, the growth factor is employed and shows its capability for inducing the differentiation of the cell.

According to the method of the present invention, the collecting unit is preferably a cylinder collecting unit for collecting the hollow conduits and is able to slightly arrange the hollow conduits simultaneously. Hence, an additional aligning step, after collection, can be eliminated. Preferably, a rotating motor may be further attached to the collector in order to control the rotating speed of the collecting unit.

Another object of the present invention is to provide a novel nerve guide conduit whereby the disadvantages of the conventional nerve guide conduit, such as low surface area, low porosity, huge diameter, unfavorable texture, long production time and high-manufacturing cost, can be overcome. The nerve guide conduit of the present invention has suitable hardness, flexibility, and large surface area for the growth of the cells.

The nerve guide conduit of the present invention comprises a plurality of hollow conduits, wherein the hollow conduits are made of a biodegradable/biocompatible material and the hollow conduits are arranged parallelly to each other. According to the nerve guide conduit of the present invention, several hollow conduits contained therein are parallely arranged, those hollow conduits being shaped as tubes comprising through channels in its long axis direction thus providing a large surface area for the growth of the cells, which cannot be obtained from the conventional nerve guide conduit. Further, since a biodegradable/biocompatible material is used as the material of the hollow conduits, nerve guide conduits constructed from the hollow conduits are certainly biodegradable/biocompatible in character.

According to the nerve guide conduit of the present invention, the biodegradable or the biocompatible material preferably is, but is not limited to, polylactic acid (PLA), polyglycolic acid (PGA), poly(lactic-co-glycolic acid) (PLGA), polycaprolactone (PCL), collagen, chitosan, polyalkyl acid, alginate, polyamide, or the combinations thereof.

According to the nerve guide conduit of the present invention, each of the hollow conduits can be made from any possible method, but are preferably made by electrospinning.

Preferably, according to the nerve guide conduit of the present invention, a supporting solution is further comprised in the through channels of the hollow conduits, but is not limited thereto.

According to the nerve guide conduit of the present invention, the supporting solution preferably is, but is not limited to, a solution of poly vinyl pyrrolidone (PVP), poly ethylene oxide (PEO), poly ethylene glycol (PEG), or the combinations thereof.

Preferably, according to the nerve guide conduit of the present invention, at least one cell is further contained in the hollow conduits of the nerve guide conduit, wherein the cell is preferably a nerve regeneration cell, but is not limited thereto.

According to the nerve guide conduit of the present invention, the nerve regeneration cell preferably is, but is not limited to, a neural stem cell, Schwann cell, Satellite Cells, oligodendrocyte, astrocyte, microglia, ependymal cells, or the combinations thereof.

According to the nerve guide conduit of the present invention, at least a growth factor may be comprised in the hollow conduits of the nerve guide conduit to induce the differentiation of the cell.

Therefore, the nerve guide conduit of the present invention can meet the six specific requirements for being an excellent nerve guide conduit, which are porosity/biodegradability, support cell incorporation, piezoelectricity, growth factor release control, oriented nerve substratum inclusion, and intraluminal channels containment. The nerve guide conduit of the present invention has suitable hardness, flexibility, and large surface area for the growth of the cells. Hence, the disadvantages of the conventional nerve guide conduit, such as low surface area, low porosity, huge diameter, unfavorable texture, long production time and high-manufacturing cost, can be overcome by the present invention.

Other objects, advantages, and novel features of the invention will become more apparent from the following detailed description when taken in conjunction with the accompanying drawings.

### BRIEF DESCRIPTION OF THE DRAWINGS

FIG. 1 is a process flow chart for electrospinning to prepare a nerve guide conduit of the Example 1; and

FIG. 2 is a process flow chart for electrospinning to prepare a nerve guide conduit of the Example 1.

### DETAILED DESCRIPTION OF THE PREFERRED EMBODIMENT

### EXAMPLE 1

### Solution Preparation and Fabrication of Hollow Fibers Scaffold by Electrospinning

Solutions for electrospinning were (1) 10 wt % Poly-L-lactic acid (PLLA, medical grade, Mw=140 kDa, kindly supplied by BioTechOne Inc. Taiwan) in mixed N,N-Dimethyl formamide /Dichloromethane solution (DMF, HCON(CH₃)₂, 99.8%, Tedia, USA/DCM, CH₂Cl₂, reagent grade, 99.9%, Mallinckrodt, USA) and (2) Poly-ethylene glycol/Poly-ethylene oxide (PEG, Mw=35 kD, PEO, Mw=900 kD, both from Sigma-Aldrich, USA) 10 wt % aqueous solution. The electrospinning setup consisted of a static charger (SIMCO, CD50-P, Chargermaster, USA) 4, two syringe pumps (KDS-100, USA)13,14 and collecting unit 2, either a metal flat plate, or a rotating drum with a diameter of 7 cm.

The electrospinning processes were carried out in conjunction with a core/shell spinneret 10 to produce core/shell fibers with the following parameters: up to 20 kV of applied voltage and 10 to 20 centimeter of collecting distance.

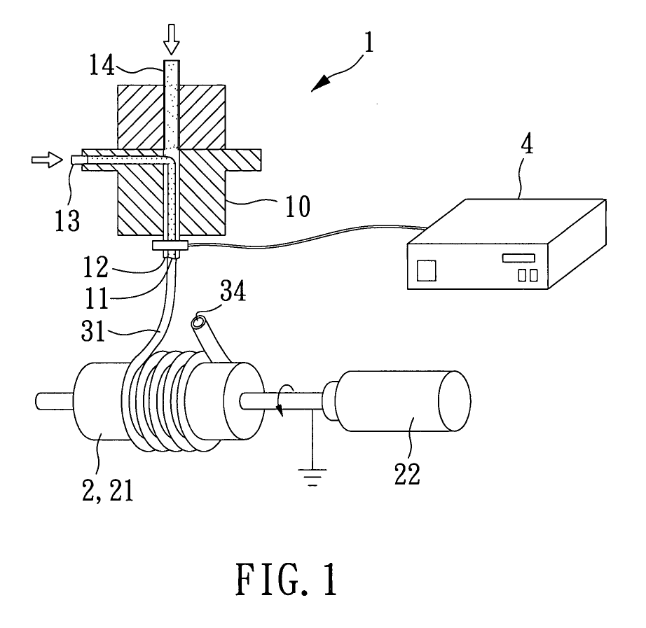

In reference to FIG. 1, a process flow chart for electrospinning to prepare a nerve guide conduit of the present invention is shown. First, (A) an electrospinning device 1 having a core/shell spinneret 10, a first syringe pump 13, a second syringe pump 14 and a collecting unit 2 is prepared. The core/shell spinneret 10 has an inner outlet 11 and an outer outlet 12 such that the inner outlet 11 and the outer outlet 12 are coaxial, the diameter of the inner outlet 11 is 0.9 mm, and the diameter of the outer outlet 12 is 1.4 mm. Herein, the first syringe pump 13 connected to the inner outlet 11, and the second syringe pump 14 connected to the outer outlet 12. The collecting unit 2 is a cylindrical collecting unit 21 connecting to a rotating motor 22 that is used for controlling the rotating rate of the cylindrical collecting unit 21. In addition, a static charger (SIMCO, CD50-P, Chargermaster, USA) 4 is used to provide a drawing force during electrospinning. Subsequently, (B) the second syringe pump 14 is filled with a PLLA (poly-L lactic acid) solution, and first syringe pump 13 is filled with a PEG/PEO (poly ethylene glycol/poly ethylene oxide) solution. (C) The voltage is set to 20 kV. Electrospinning is performed and the hollow conduits 31 outputs from the core/shell spinneret 10 are collected by the collector 2 with a gap of 10 to 20 cm and then collected by the collecting unit 2. Herein, the conduits 31 are presented as tubes comprising through channels 34, in which the walls of the conduits 31 are constructed by the PLLA molecules and the through channels 34 of the tubes are filled with PEG/PEO molecules. In this step, changing the flow rate of the syringes pump 13,14 may obtain different diameters of the hollow conduits 31, and the diameters of the hollow conduits 31 should be 100 μm or less.

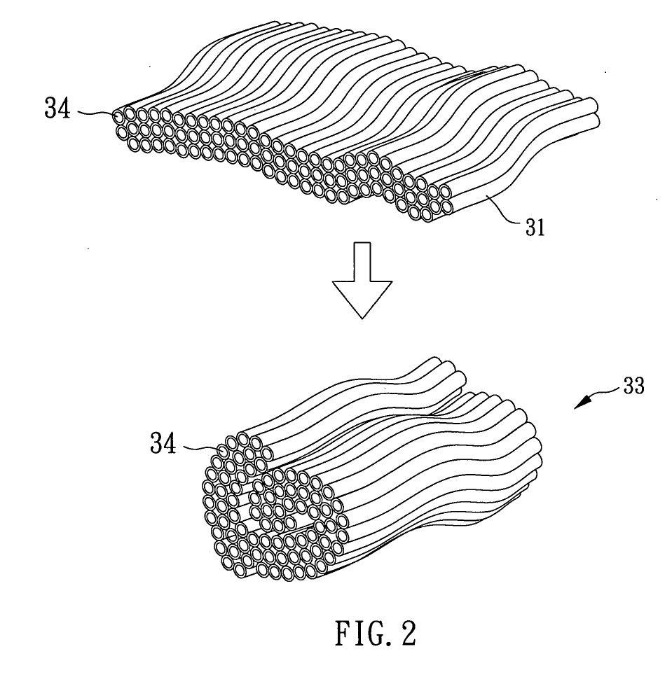

Then, (C1) washes out the PEG/PEO molecules from the through channels 34 of the hollow conduits 31 with water for 48 hours. After removing the extra solvent in the inner tube by drying, hollow conduits 31 (hollow fibers) were obtained. Those hollow conduits 31 are then parallelly aligned. Finally, with reference of FIG. 2, (D) the hollow conduits 31 are curled by hand to produce a bundle as the nerve guide conduit 33 of the present invention.

By adjusting processing parameters, such as, voltage and relative flow rates of the outer PLA solution, and inner PEO-PEG aqueous solution, the electrospinning processes were conducted and core/shell fibers were collected either by a rotating device or a flat plate. Micron scale hollow fibers were obtained after drying the core/shell fibers prepared by following parameters: 20 kV, 15 cm, and 1.5 and 1.2 ml/hr for outer and inner flow rates, respectively. It was found that the relative flow rate played an important role regarding the successful formation of the core/shell fibers. While the solution was pushed out from the spinneret it tended to break easily if the relative flow rate and viscosity were poorly matched. Early morphological characterization has found the physical dimension of the fibers were from 20 to 70 microns in diameter. Another morphological characteristic we observed was the co-existence of significantly large, hollow fibers with a minor amount of small, solid ones, some with dimensions down to several hundred nanometers. Unlike the larger, oriented hollow fibers, the smaller ones were distributed randomly among the larger fibers without a preferred direction, even with rotating collections. The cross section view revealed hollow structures with wall thicknesses around a few microns. We also observed, under certain spinning conditions, an interesting micro-porous structure on the fiber wall.

With the major goal of producing smaller hollow fibers in mind, we successfully prepared hollow fibers of 20-60 microns in diameter, a significant reduction compared to those reported from previous works. With the reduction in diameter, an estimated 5 to 10 fold increase of surface area could be achieved for more cell attachment and hence, a better neurite connection environment. Also, an interesting micro-porous structure of the fiber wall was observed, which might be a significant advantage as a source for nutrient permeation and/or metabolic waste disposal, as suggested by several researchers. The formation of this wall structure may well be due to the solvent employed in the electrospinning process. It was observed previously that fast solvent vaporization during the process could generate such porous wall. We also tried to achieve similar structures via different schemes, e.g. by leaching out the pore-genic materials added within polymer. With the addition of the water soluble particle, such as glucose, we were able to make the fiber walls porous right after the hollow fibers were collected. One major advantage of this approach is the better control of the pore size by selecting different sizes of these pore-genic particles. The observed smaller, randomly distributed fibers were also reported in other research and believed to have formed by a smaller jet that randomly side-swung from the main jet, hence collected without preferred direction, during the electrospinning process. We also proved that all these smaller fibers were PLA, by comparing the dimensions of them before and after washing with water. No noticeable diameter changes were observed, which would indicate the above mentioned situation of smaller PLA fiber formation. Structure-wise, in our case, these smaller, random fibers may have severed as connection strings for holding together the larger, oriented fibers. The wide angle X-ray diffraction pattern showed that the fibers consisted of mainly PLA with a trace of PEG. The DSC data also echoed this finding.

The method of fabricating nerve guide conduit of the present invention uses an electrospinning process to provide hollow conduits, followed by curling to form bundles to give the nerve guide conduit. The method of the present invention has many advantages including short processing time and high manufacturing efficiency. The method of the present invention is the first one applying electrospinning into the manufacturing of multi-tubular nerve guide conduit. Besides, the biodegradable/biocompatible nerve guide conduit fabricated by the present invention has large surface area for cell guiding/growth and has suitable hardness and flexibility. Therefore, the disadvantages of the conventional nerve guide conduit, such as low surface area, low porosity, huge diameter, unfavorable texture, long production time and high-manufacturing cost, can be overcome by using the method of fabricating nerve guide conduits of the present invention. Moreover, the method of the present invention has the potential of generating multifunctional conduits by using a simple process.

Meanwhile, with the rearrangement of molecules and the improved crystallinity of the materials after electrospinning, further with the characteristic of the material of polylactic acid (PLA) itself, the nerve guide conduit of the present invention may have specific piezoelectricity. Therefore, an inner stimulation such as diameter change or an outer stimulation such as ultrasonic waves may enhance electric current for axon growth guidance/induction.

### EXAMPLE 2

Cell Culture

PC12 cells were obtained from ATCC (CRL-1721, HisnChu Food Industrial Research Center, Taiwan). Prior to the electrospinning process, PC12 cells were maintained in a DMEM medium supplemented with 10% fetal bovine serum (Biological Industries, Israel), 50 U/ml penicillin, and 50 mg/ml streptomycin (full medium, Biological Industries, Israel). Cells were routinely sub-cultured every 5-6 days. Neuronal differentiation of the PC12 cells was carried out by adding nerve growth factor (7.5 s mouse 50 ng/ml, NGF-7S, Sigma, USA) into DMEM with 1% FBS for the required time. Right before the electrospinning process, the cells and medium were added into the PEO/PEG solution and mixed for 10 min.

Preparation of Fluorescent PC12 Cells

PC12 cells were transfected with a pEGFP-N1 (Clontech) construct, which expresses a green fluorescent protein. Seventy microliters of Lipofectamine2000 (Invitrogen) and 25 μg of pEGFP-N1 DNA were mixed in 3 ml of OPTI-MEM and incubated for 20 minutes with gentle shaking at room temperature. The Lipofectamine 2000 and DNA mixture were then added to ˜2×10⁶ PC12 cells with 3 ml of OPTI-MEM in a T75 cultural flask, which was pre-coated with poly-L-lysine. The media was swirled to ensure even coverage and the cells were incubated at 37° C., 5% CO_{2.} Six hours later, the transfection mixture was replaced with DMEM containing 1% fetal bovine serum and antibiotics, continuously cultured at 37° C., 5% CO₂ for 24-48 hrs before being readied for the next experiments.

Bio-Electrospinning

In a clean room, bio-electrospinning was conducted in a similar set-up and fashion to the normal electrospinning process mentioned above. After proper sterilization steps were taken, the cells in the medium, 10⁶ x/ml, were added to PEO/PEG 10 wt % aqueous solution and mixed well before being transferred to the syringe pump. The electrospinning processes were carried out with parameters similar to previous one. The obtained cell-containing fibers were placed in a cell culture medium after removal from the collecting unit for further observation.

Bio-Electrospinning, Cells Inside the Hollow Fiber

The hollow fibers collected via the bio-electrospinning process were cultured in the medium. With aid of the optical microscope, it was found that cells were floating and moving inside of the hollow fiber right after the formation of these fibers. The finding was confirmed with the fluorescent microscopy with DNA transfected PC12 cells. A few days after the addition of the NGF, either right before the spinning process or in the culture solution of cell-containing hollow fiber, PC-12 cell attached and transformed from a round shape to a more elongated one, indicating the attachment of the cell. In the Day 5 to 6 period, neurite extension of up to 3-5 times the original body size was also observed, some as long as 100 microns. In some cases, the growth cone at the end of neurite was clearly seen. The growth direction of the neurite was the same as that of the fiber. These observations were confirmed with the DNA modified PC-12 cell under a fluorescent microscope.

According to the SEM photograph results, PC12 cells were found to be alive inside the tube for more than a week under appropriate culture conditions. Several conclusions can be made from this observation. First, it was suggested that cells, at least some of them, could survive electrospinning, both the high voltage application and possible solvent contact. This can be explained, as also demonstrated by other research groups with other cells in similar conditions, as being due to extremely short time exposure to such conditions, as suggested by Jayasinghe et al. However, at this moment, no quantified data could be obtained due to several challenges to the experimental design. For example, the viability of cell could not be easily measured by a traditional assay. The agent needed to dye the dead cell may not be easily reached within the hollow fibers. The dissolution of the PLA hollow fibers could be accomplished by adding the appropriate solvent, such as dichloromethane; however, should the cell be affected by this process, it may cause the low viability measurements. As for other important components needed in the regenerative process, such as growth factors and the extracellular matrix or substratum proteins with which cells interact, there were reports discussed the effect of electrospinning on their functions. Koh et al demonstrated that the Laminin still had the capability to aid cell attachment and differentiation even after it was electrospun. Chew reported similar results on the protein bioactivity upon electrospinning process and the bioactivity of the NGF was sustained, if not completely, after electrospinning process. As shown in our results, the observed neurite outgrowth toward the direction of the fiber clearly demonstrates the guiding function of the hollow fiber. With the above results, i.e. larger surface area, higher degree of orientation, porous walls, and biocompatible guiding structures, we demonstrate a novel bio-electrospinning process for creating a multi-functional scaffold for nerve guide conduits via selected materials.

Aligned, micro-scale tubular, cell-containing scaffolds were prepared via a novel bio-electrospinning process with the biodegradable polymer. An advanced nerve guide conduit was then easily prepared with the combination of the all three major elements of tissue engineering. The PC-12 cells were introduced in the tubular scaffold simultaneously and showed their attachment, proliferation, and finally differentiation, with the addition of the neuron growth factor. The neurite of PC-12 cell was observed extending along the direction of the micro-tubular scaffold. This data showed the viability of the PC-12 cell and nerve growth factor to retain certain capabilities after the electrospinning process and demonstrated the future application of this process. In the meantime, a nerve guide conduit combined with most the advanced features was easily prepared.

Although the present invention has been explained in relation to its preferred embodiment, it is to be understood that many other possible modifications and variations can be made without departing from the scope of the invention as hereinafter claimed.

What is claimed is:

1. A method of fabricating a nerve guide conduit, comprising:
(A) providing (preparing) an electrospinning device, wherein the electrospinning device comprises:
a core/shell spinneret having an inner outlet and an outer outlet with that the inner outlet and the outer outlet are coaxial;
a first syringe pump connecting to the inner outlet of the core/shell spinneret;
a second syringe pump connecting to the outer outlet of the core/shell spinneret; and
a collecting unit;
(B) feeding the second syringe pump with a first material, and feeding the first syringe pump with a supporting solution;
(C) electrospinning the first material and the supporting solution by using the electrospinning device to extrude a plurality of hollow conduits, with the supporting solution inside, out from the core/shell spinneret;
(D) collecting the hollow conduits by the collecting unit, and arranging the hollow conduits parallelly; and
(E) curling up the hollow conduits to provide a nerve guide conduit;
wherein the first material is a biodegradable material or a biocompatible material.

2. The method of fabricating a nerve guide conduit as claimed in claim 1, further comprising a step (C1), after step (C), of washing the hollow conduits with a solvent.

3. The method of fabricating a nerve guide conduit as claimed in claim 2, wherein the supporting solution is washed out from the hollow conduits in the step (C1).

4. The method of fabricating a nerve guide conduit as claimed in claim 1, wherein the first material is polylactic acid (PLA), polyglycolic acid (PGA), poly(lactic-co-glycolic acid) (PLGA), polycaprolactone (PCL), collagen, chitosan, polyalkyl acid, alginate, polyamide, or the combinations thereof.

5. The method of fabricating a nerve guide conduit as claimed in claim 1, wherein the supporting solution is a solution of poly vinyl pyrrolidone (PVP), poly ethylene oxide (PEO), poly ethylene glycol (PEG), or the combinations thereof.

6. The method of fabricating a nerve guide conduit as claimed in claim 2, wherein the solvent for washing the hollow conduits is water.

7. The method of fabricating a nerve guide conduit as claimed in claim 1, wherein a cell is further contained in the supporting solution.

8. The method of fabricating a nerve guide conduit as claimed in claim 7, wherein the cell is a nerve regeneration cell.

9. The method of fabricating a nerve guide conduit as claimed in claim 8, wherein the nerve regeneration cell is a neural stem cell, Schwann cell, Satellite Cells, oligodendrocyte, astrocyte, microglia, ependymal cells, or the combinations thereof.

10. The method of fabricating a nerve guide conduit as claimed in claim 1, further comprising a growth factor added into the first material in the step (B).

11. The method of fabricating a nerve guide conduit as claimed in claim 1, wherein the collecting unit is a cylinder collecting unit.

12. A nerve guide conduit comprising a plurality of hollow conduits, wherein each of the hollow conduits independently comprises a through channel in its long axis direction, the hollow conduits are made of a first material, which is a biodegradable material or a biocompatible material, and the hollow conduits are arranged parallelly to each other.

13. The nerve guide conduit as claimed in claim 12, wherein the first material is polylactic acid (PLA), polyglycolic acid (PGA), poly(lactic-co-glycolic acid) (PLGA), polycaprolactone (PCL), collagen, chitosan, polyalkyl acid, alginate, polyamide, or the combinations thereof.

14. The nerve guide conduit as claimed in claim 12, wherein the plurality of hollow conduits are made by electrospinning.

15. The nerve guide conduit as claimed in claim 12, wherein a supporting solution is further comprised in the through channels of the hollow conduits.

16. The nerve guide conduit as claimed in claim 15, wherein the supporting solution is a solution of poly vinyl pyrrolidone (PVP), poly ethylene oxide (PEO), poly ethylene glycol (PEG), or the combinations thereof.

17. The nerve guide conduit as claimed in claim 12, wherein at least one cell is further contained in the hollow conduits of the nerve guide conduit.

18. The nerve guide conduit as claimed in claim 17, wherein the at least one cell is a nerve regeneration cell.

19. The nerve guide conduit as claimed in claim 18, wherein the nerve regeneration cell is a neural stem cell, Schwann cell, Satellite Cells, oligodendrocyte, astrocyte, microglia, ependymal cells, or the combinations thereof.

20. The nerve guide conduit as claimed in claim 12, further comprising a growth factor locating in the hollow conduits of the nerve guide conduit.
